(19) United States
(12) Patent Application Publication
(10) Pub. No.: US 2025/0018146 A1
(43) Pub. Date: Jan. 16, 2025

(54) INFUSION SOLUTION OXYGENATION DEVICE AND INFUSION SOLUTION OXYGENATION SYSTEM

(71) Applicant: TERUMO KABUSHIKI KAISHA, Tokyo (JP)

(72) Inventors: Satoshi SAWADA, Hadano city (JP); Yu OSAWA, Ebina-city (JP)

(73) Assignee: TERUMO KABUSHIKI KAISHA, Tokyo (JP)

(21) Appl. No.: 18/899,321

(22) Filed: Sep. 27, 2024

Related U.S. Application Data
(63) Continuation of application No. PCT/JP2023/008908, filed on Mar. 8, 2023.

(30) Foreign Application Priority Data
Mar. 29, 2022 (JP) 2022-054668

(51) Int. Cl. A61M 25/00

(52) U.S. Cl. CPC A61M 25/00; A61M 2202/0476

(57) ABSTRACT

An infusion solution oxygenation device and an infusion solution oxygenation system capable of supplying oxygen to an infusion solution safely, simply, and quickly. An infusion solution oxygenation device includes a catheter connection portion to which a catheter that supplies an oxygen-containing gas to an infusion solution in an infusion solution container is connected, a gas supply unit that supplies the oxygen-containing gas to the catheter connection portion, and a container pressure sensing unit that senses an internal pressure of the infusion solution container, the gas supply unit controlling the supply of the oxygen-containing gas on the basis of the internal pressure of the infusion solution container sensed by the container pressure sensing unit.

### CROSS-REFERENCES TO RELATED APPLICATIONS

This application is a continuation of International Application No. PCT/JP2023/008908 filed on Mar. 8, 2023, which claims priority to Japanese Application No. 2022-054668 filed on Mar. 29, 2022, the entire content of both of which is incorporated herein by reference.

### TECHNOLOGICAL FIELD

The present disclosure generally relates to an infusion solution oxygenation device and an infusion solution oxygenation system.

### BACKGROUND DISCUSSION

Japanese Patent Application Publication No. 2006/263018 A discloses an infusion solution line monitoring system. This infusion solution line monitoring system detects a power supply frequency signal induced by capacitance formed between a power supply line and a living body by an electrode attached to the infusion solution line. The infusion solution line includes a tube, a catheter, and the like through which a medicinal solution or blood flows. A needle or a thin tube is provided at one end of the infusion solution line, and it is possible to cause an infusion solution such as a medicinal solution or a liquid to flow into the living body by inserting the needle or the thin tube into a desired position of the living body. The other end of the infusion line is connected to the medicinal solution bottle. An electrode is provided in the middle of the path of the infusion solution line.

Japanese Patent Application Publication No. 2019-500195 A discloses a system and method for directly delivering a liquid containing a dissolved medical gas via an arterial system, and a system and method for reducing the harm of inadvertent injection of air during a medical procedure, particularly when flushing a line with the fluid prior to insertion into the body, for delivering a medical liquid containing a gas. The system includes a medical gas cylinder with a regulator, a vacuum pump, a three-way valve with a solenoid, a controller coupling to a solenoid, a heat-insulated container, a collapsible fluid tank with a gas/physiological layer, a three-way valve, a fluid in-line sensor, e.g., a fluid in-line sensor, one coupled with a line adjacent to the three-way valve and a second portion coupled with a fluid path to a catheter adjacent to a catheter that is distal to the fluid tank, a controller including a power source, an ultrasonic processor connected to the controller, a heater/cooler including a temperature sensor and a coupling from the solenoid to a computer via a coupling to the controller, a three-way valve with a solenoid, and a combination of fluid paths to a catheter that may include a connector to the catheter system. In this system, as an example, oxygen is dissolved in saline solution, and the saline solution can be used as an oxygen carrier to supply oxygen to a patient by drip of the saline solution or the like.

Japanese Patent Application Publication No. 2007-289695 A discloses an oxygenator having a gas exchange membrane that separates a blood side and a gas side.

In a living body, when the arterial oxygen saturation (so-called saturation of percutaneous oxygen (SpO2), also referred to as blood oxygen saturation) decreases, the amount of oxygen supplied to the periphery decreases. As a result, the peripheral tissue falls into a hypoxic state. In this manner, in a state where the pulmonary function is insufficient, treatment such as oxygen supply through the lungs such as oxygen concentration adjustment of a respiratory gas, positive pressure oxygen therapy, and use of a ventilator, and oxygen supply by an extracorporeal circulation device (see, for example, Japanese Patent Application Publication No. 2007-289695 A) using an oxygenator and a blood pump is performed. However, oxygen delivery via the lungs may not provide a sufficient effect when the gas exchange capacity in the lungs is reduced. In addition, when it is necessary to install the tracheal cannula, there is a problem that invasion cannot be avoided, and it takes time and effort. The use of the extracorporeal circulation device requires cooperation of a plurality of doctors, engineers, nurses, and the like, and it takes much time and effort to use the extracorporeal circulation device. In addition, the extracorporeal circulation device may not be promptly ready for use, and it may take time to start use after starting installation. Therefore, prompt treatment for the patient may not be possible.

Oxygen delivery to the patient via infusion solution (see, for example, Japanese Patent Application Publication No. 2019-500195 A) can potentially provide a less invasive approach. In addition, if an infusion solution in which oxygen is dissolved can be prepared, there is a possibility that oxygen can be quickly supplied to the patient. However, a method for safely and easily and quickly dissolving oxygen in an infusion solution is not sufficiently provided. Therefore, it is desired to provide a device that can safely and easily and quickly supply oxygen to an infusion solution.

### SUMMARY

The present disclosure has been made in view of such an actual situation, and provides an infusion solution oxygenation device and an infusion solution oxygenation system capable of supplying oxygen to an infusion solution safely, simply, and quickly.

An infusion solution oxygenation device according to the present disclosure includes: a catheter connection portion to which a catheter that supplies an oxygen-containing gas to an infusion solution in an infusion solution container is connected; a gas supply unit that supplies the oxygen-containing gas to the catheter connection portion; and a container pressure sensing unit that senses an internal pressure of the infusion solution container, in which the gas supply unit controls supply of the oxygen-containing gas based on the internal pressure sensed by the container pressure sensing unit.

In the infusion solution oxygenation device according to the present disclosure, the catheter connection portion may further include: an air supply unit that supplies the oxygen-containing gas into the infusion solution container; and an exhaust unit that exhausts gas from the inside of the infusion solution container.

The infusion solution oxygenation device according to the present disclosure may further include an oxygen concentration sensing unit that senses an oxygen concentration of the infusion solution, in which the gas supply unit may control the supply of the oxygen-containing gas based on the oxygen concentration sensed by the oxygen concentration sensing unit.

The infusion solution oxygenation device according to the present disclosure may further include a pressure adjustment unit that adjusts an internal pressure of the infusion solution container, in which the pressure adjustment unit may adjust the internal pressure based on the oxygen concentration sensed by the oxygen concentration sensing unit.

The infusion solution oxygenation device according to the present disclosure may further include a biological information acquisition unit that acquires biological information of a patient, in which the gas supply unit controls the supply of the oxygen-containing gas based on the biological information acquired by the biological information acquisition unit.

In the infusion solution oxygenation device according to the present disclosure, the biological information may further include a blood oxygen saturation, an inhaled oxygen concentration, a blood pressure, a heart rate, a respiratory rate, a tissue oxygen saturation, an arterial oxygen partial pressure, a mixed venous blood oxygen saturation, a urine oxygen partial pressure, an intravesical pressure, a brain/tissue oxygen saturation, a urine amount, a body temperature, or a cardiac output.

The infusion solution oxygenation device according to the present disclosure may further include a remaining amount sensing unit that senses a remaining amount of the infusion solution in the infusion solution container, in which the gas supply unit may control the supply of the oxygen-containing gas based on the remaining amount of the infusion solution acquired by the remaining amount sensing unit.

The infusion solution oxygenation device according to the present disclosure may further include a temperature sensing unit that senses a temperature of the infusion solution, in which the gas supply unit may control the supply of the oxygen-containing gas based on the temperature sensed by the temperature sensing unit.

The infusion solution oxygenation device according to the present disclosure may further include: an oxygen concentration sensing unit that senses an oxygen concentration of the infusion solution; a temperature sensing unit that senses a temperature of the infusion solution; and a temperature adjustment signal transmission unit that transmits a temperature adjustment signal for adjusting the temperature of the infusion solution, in which the temperature adjustment signal transmission unit may transmit the temperature adjustment signal based on the temperature sensed by the temperature sensing unit or the oxygen concentration sensed by the oxygen concentration sensing unit.

The infusion solution oxygenation device according to the present disclosure may further include: an oxygen concentration sensing unit that senses an oxygen concentration of the infusion solution; a pressure adjustment signal transmission unit that transmits a pressure adjustment signal for adjusting an internal pressure of the infusion solution container, in which the pressure adjustment signal transmission unit may transmit the pressure adjustment signal based on the internal pressure sensed by the container pressure sensing unit or the oxygen concentration sensed by the oxygen concentration sensing unit.

The infusion solution oxygenation device according to the present disclosure may further include: a first connection portion connected to a supply source of a first gas containing an oxygen gas; and a second connection portion connected to a supply source of a second gas different from the first gas, in which the second gas may contain nitrogen, argon, nitrogen monoxide, nitrous oxide gas, or carbon dioxide, and the gas supply unit may prepare the oxygen-containing gas by mixing the first gas and the second gas.

An infusion solution oxygenation system according to the present disclosure for achieving the above object includes: a catheter that supplies an oxygen-containing gas to an infusion solution in an infusion solution container; a gas supply unit that supplies the oxygen-containing gas to the catheter; and a container pressure sensing unit that senses an internal pressure of the infusion solution container, in which the gas supply unit controls supply of the oxygen-containing gas based on the internal pressure sensed by the container pressure sensing unit.

In the infusion solution oxygenation system according to the present disclosure, the catheter may further include: a supply lumen that supplies the oxygen-containing gas into the infusion solution container; and an exhaust lumen that exhausts gas from the inside of the infusion solution container.

A method is disclosed for supplying to an infusion solution, the method including: supplying an oxygen-containing gas to an infusion solution in an infusion solution container via a catheter, the catheter being connected to a catheter connection portion; supplying the oxygen-containing gas to the catheter connection portion via a gas supply unit; sensing an internal pressure of the infusion solution container with a container pressure sensing unit; and controlling the supply of the oxygen-containing gas by the gas supply unit based on the internal pressure sensed by the container pressure sensing unit.

It is thus possible to provide an infusion solution oxygenation device and an infusion solution oxygenation system capable of supplying oxygen to an infusion solution safely, simply, and quickly.

### BRIEF DESCRIPTION OF THE DRAWINGS

FIG. 1 is a diagram for explaining a configuration of an infusion solution oxygenation device according to a first embodiment and an infusion solution oxygenation system provided with the same.

FIG. 2 is a functional block diagram of the infusion solution oxygenation device according to the first embodiment.

FIG. 3 is an example of a use state of the infusion solution oxygenation device and the infusion solution oxygenation system provided with the same.

FIG. 4 is a diagram for explaining a structure of a catheter.

FIG. 5 is a cross-sectional view taken along a line V-V of FIG. 4.

FIG. 6 is a diagram illustrating an example of a display on a display unit.

FIG. 7 is a diagram illustrating an example of a display on the display unit.

FIG. 8 is a diagram illustrating an example of a display on the display unit.

FIG. 9 is a graph showing results of animal experiments.

FIG. 10 is a diagram for explaining a configuration of an infusion solution oxygenation device according to a second embodiment and an infusion solution oxygenation system provided with the same.

FIG. 11 is a diagram for explaining a configuration of an infusion solution oxygenation device according to a third embodiment and an infusion solution oxygenation system provided with the same.

FIG. 12 is a diagram for explaining a configuration of an infusion solution oxygenation device according to a first modification.

FIG. 13 is a functional block diagram of an infusion solution oxygenation device according to second and third modifications.

FIG. 14 is a diagram for explaining a configuration of an infusion solution oxygenation device according to another embodiment and an infusion solution oxygenation system provided with the same.

FIG. 15 is a diagram for explaining a configuration of an infusion solution oxygenation device according to another embodiment and an infusion solution oxygenation system provided with the same.

### DETAILED DESCRIPTION

An infusion solution oxygenation device and an infusion solution oxygenation system according to an embodiment of the present disclosure will be described with reference to the drawings.

### First Embodiment

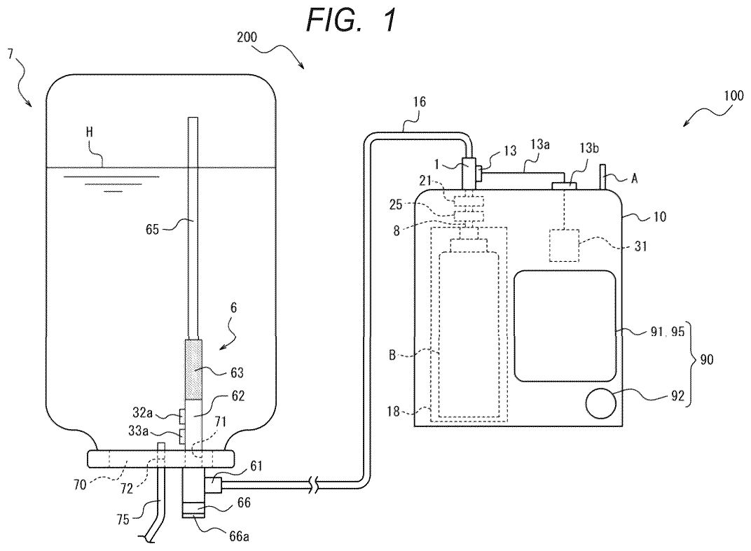

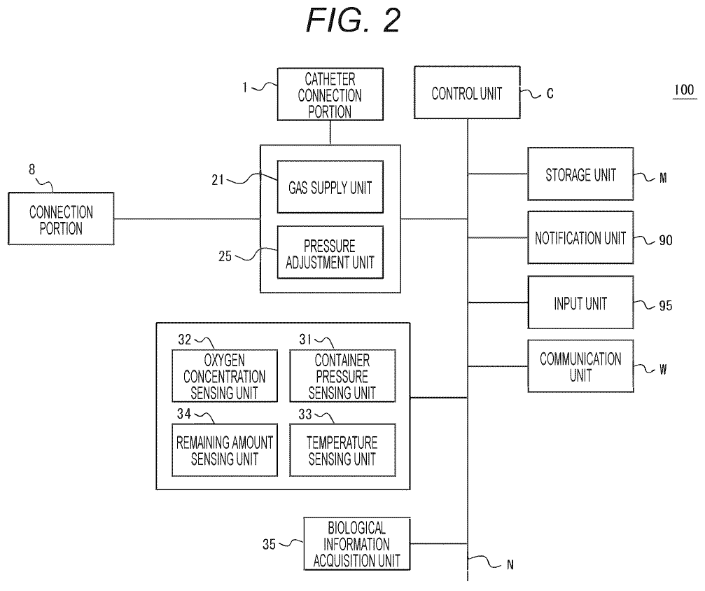

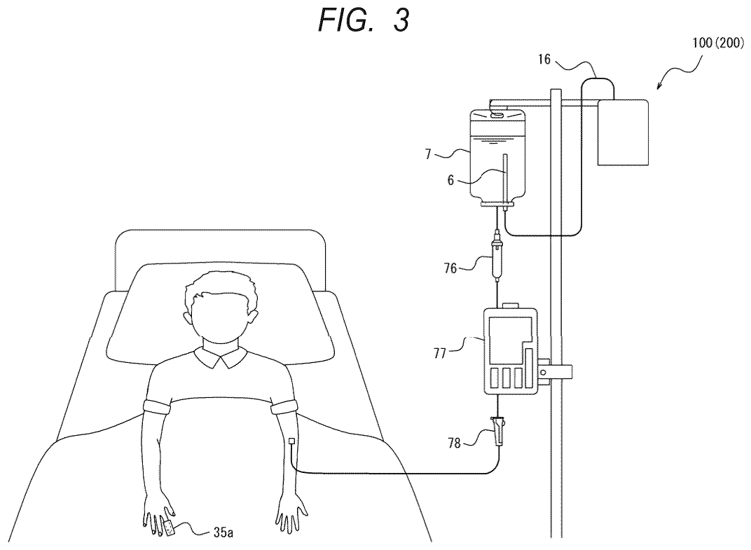

FIG. 1 illustrates an infusion solution oxygenation system 200 including an infusion solution oxygenation device 100 according to the present embodiment. FIG. 2 shows a functional block diagram of the infusion solution oxygenation device 100. FIG. 3 shows an example of a use situation of the infusion solution oxygenation device 100 and the infusion solution oxygenation system 200.

As illustrated in FIG. 1, the infusion solution oxygenation system 200 can include the infusion solution oxygenation device 100 and a catheter 6 that is connected to the infusion solution oxygenation device 100 and supplies an oxygen-containing gas to the infusion solution in an infusion solution container 7.

The infusion solution oxygenation device 100 includes a catheter connection portion 1 to which the catheter 6 that supplies an oxygen-containing gas to an infusion solution in the infusion solution container 7 is connected, a gas supply unit 21 that supplies the oxygen-containing gas to the catheter connection portion 1, and a container pressure sensing unit 31 that senses an internal pressure of the infusion solution container 7 (hereinafter, simply referred to as internal pressure).

The gas supply unit 21 controls the supply of the oxygen-containing gas on the basis of the internal pressure of the infusion solution container 7 sensed by the container pressure sensing unit 31.

The infusion solution oxygenation device 100 and the infusion solution oxygenation system 200 using the same can safely and simply and quickly supply oxygen to the infusion solution.

As illustrated in FIG. 1, the infusion solution oxygenation system 200 includes the infusion solution oxygenation device 100 and the catheter 6. The catheter 6 is used in a state of being inserted into the infusion solution container 7 such as a drip bag.

First, the overall configurations of the infusion solution oxygenation system 200 and the infusion solution oxygenation device 100 will be described.

The infusion solution oxygenation device 100 includes a housing 10, the catheter connection portion 1 to which the catheter 6 is connected, the gas supply unit 21 that supplies an oxygen-containing gas to the catheter connection portion 1, and the connection portion 8 connected to a supply source of the oxygen-containing gas (for example, an oxygen cylinder B). The infusion solution oxygenation device 100 supplies the oxygen-containing gas supplied from the oxygen cylinder B to the catheter 6 via the catheter connection portion 1. The catheter 6 releases and supplies the oxygen-containing gas in the infusion solution in the infusion solution container 7 to dissolve oxygen in the infusion solution.

Hereinafter, dissolving oxygen in the infusion solution to increase the oxygen concentration of the infusion solution may be simply referred to as oxygenation or the like. For example, in a case where it is described that the infusion solution is oxygenated, it means that oxygen is supplied to and dissolved in the infusion solution to increase the oxygen concentration of the infusion solution.

The infusion solution oxygenation device 100 may be used in a state of being suspended from, for example, an infusion stand. The infusion solution oxygenation device 100 may have a locking portion locked to the infusion stand in the housing 10 (see FIG. 3). Alternatively, a locking portion that is connected to and fixed to a bed on which a patient lies, another medical device may be provided, or an independent stand may be provided.

The infusion solution oxygenation device 100 includes a container pressure sensing unit 31 that senses the internal pressure of the infusion solution container 7 (hereinafter simply referred to as internal pressure) in order to ensure the safety of oxygen supply to the infusion solution and the safety when the oxygen-dissolved infusion solution is administered to the patient. In the infusion solution oxygenation device 100, the supply of the oxygen-containing gas from the gas supply unit 21 is controlled on the basis of the internal pressure sensed by the container pressure sensing unit 31. As a result, for example, rupture of the infusion solution container 7 can be prevented, and a problem that the concentration of oxygen dissolved in the infusion solution becomes too low or too high can be avoided.

As illustrated in FIG. 2, the infusion solution oxygenation device 100 may include, in addition to the container pressure sensing unit 31, an oxygen concentration sensing unit 32 that senses the oxygen concentration of the infusion solution, a temperature sensing unit 33 that senses the temperature of the infusion solution, a remaining amount sensing unit 34 that senses the remaining amount of the infusion solution in the infusion solution container 7, and the like in order to grasp the state inside the infusion solution container 7, manage the infusion solution, and control these. By including the sensing unit of these parameters, safety can be improved. Furthermore, a biological information acquisition unit 35 that acquires biological information of a patient may be provided. In the present embodiment, hereinafter, a case where the infusion solution oxygenation device 100 includes the oxygen concentration sensing unit 32, the temperature sensing unit 33, the remaining amount sensing unit 34, and the biological information acquisition unit 35 will be described as an example.

The infusion solution oxygenation device 100 may include a pressure adjustment unit 25 that adjusts the internal pressure of the infusion solution container 7 in addition to the gas supply unit 21. In the present embodiment, a case where the infusion solution oxygenation device 100 includes the pressure adjustment unit 25 will be described below as an example.

The infusion solution oxygenation device 100 may include a notification unit 90 that notifies a user such as a doctor or a nurse of the operation state of the infusion solution oxygenation device 100, as well as states of the infusion solution container 7 and the infusion solution. In the present embodiment, a case where the infusion solution oxygenation device 100 includes the notification unit 90 will be described below as an example.

The infusion solution oxygenation device 100 may include an input unit 95 that receives an input of an operation command from a user. In the present embodiment, a case where the infusion solution oxygenation device 100 includes the input unit 95 will be described below as an example.

The infusion solution oxygenation device 100 may include a communication unit W that exchanges information and operation commands with an external medical device or sensor. In the present embodiment, a case where the infusion solution oxygenation device 100 includes the communication unit W will be described below as an example.

In the present embodiment, the operation of each unit of the infusion solution oxygenation device 100 may be controlled on the basis of the operation command of the control unit C. In the present embodiment, a case where the infusion solution oxygenation device 100 is controlled on the basis of the operation command of the control unit C will be described below as an example.

Hereinafter, each unit of the infusion solution oxygenation system 200 and the infusion solution oxygenation device 100 will be described.

The control unit C illustrated in FIG. 2 is a functional unit serving as a central control mechanism of the infusion solution oxygenation device 100 that controls the operation of the infusion solution oxygenation device 100. The control unit C receives information related to the operation status and the detection status from each unit of the infusion solution oxygenation device 100, external sensors, and an external device, and transmits an operation command to each unit of the infusion solution oxygenation device 100, external sensors, and an external device to control operations of the each unit of the infusion solution oxygenation device 100, the external sensors, and the external device. Specific control performed by the control unit C will be described later.

The control unit C may be realized in hardware or software by a CPU or a program (software) that causes the CPU to realize the control of the infusion solution oxygenation device 100. The control unit C may be realized by a computer such as a personal computer, a microcomputer, or a programmable logic controller (PLC), and control software stored in a storage device of personal computer, the microcomputer, or the PLC. In the present embodiment, a case where the control unit C is realized by software through execution of a program stored in the storage unit M built in the infusion solution oxygenation device 100 by a CPU built in the infusion solution oxygenation device 100 is exemplified. The control unit C and the storage unit M can be communicably connected to each unit of the infusion solution oxygenation device 100 via a communication path N including a port, a bus, a wired or wireless network (including the Internet connection), and the like. As the storage unit M, a storage device or a storage medium such as a so-called flash memory, a hard disk, a solid-state drive (SSD), or an optical disk such as a CD-ROM can be used.

The communication unit W can communicate with an external medical device or sensor (hereinafter may be referred to as an external device) by wireless communication via the antenna 19, for example, and can mediate exchange of information and operation commands between the infusion solution oxygenation device 100 and the external device. Note that the communication unit W is not limited to wireless communication, and may perform analog communication via a BNC, a pin jack, a dedicated connector, or the like, digital communication such as RS-232C or USB, or communication via a wired communication line such as a combination of an analog communication and a digital communication. Although individual exemplary description is omitted, any wireless communication in the present embodiment can be replaced with wired communication.

In the present embodiment, each unit of the infusion solution oxygenation device 100 can communicate with the external device via the communication unit W as necessary. In the following description, communication with the external device performed by each unit of the infusion solution oxygenation device 100 is mediated by the communication unit W, and individual description may be omitted.

As illustrated in FIG. 1, the catheter connection portion 1 is a connection mechanism for connecting the supply tube 16 connected to the catheter 6 to the infusion solution oxygenation device 100. The catheter connection portion 1 can be, for example, a liquid-tight joint such as a luer connector. The catheter connection portion 1 may be fixed to, for example, the housing 10.

The gas supply unit 21 is a supply control mechanism that supplies the oxygen-containing gas to the catheter 6 and controls the supply of the oxygen-containing gas. The gas supply unit 21 adjusts the amount of the oxygen-containing gas and supplies the same to the supply tube 16 connected to the catheter connection portion 1. The gas supply unit 21 may be an aperture adjusting valve capable of adjusting the supply amount of the oxygen-containing gas by adjusting the aperture, and as an example, a needle valve or a diaphragm valve may be employed. In this case, the gas supply unit 21 may be an automatic valve including an actuator. The gas supply unit 21 is supplied with the oxygen-containing gas from a supply source of the oxygen-containing gas, and supplies the oxygen-containing gas to the supply tube 16. The gas supply unit 21 can be, for example, accommodated in the housing 10.

The pressure adjustment unit 25 is a pressure adjusting mechanism that adjusts the internal pressure of the infusion solution container 7. In the present embodiment, as an example, a case has been described in which the pressure adjustment unit 25 adjusts the pressure of the oxygen-containing gas supplied from the catheter 6 to the infusion solution container 7 by supplying the oxygen-containing gas with the adjusted pressure to the gas supply unit 21, thereby adjusting the internal pressure of the infusion solution container 7. The pressure adjustment unit 25 may be a pressure adjustment valve capable of adjusting or controlling the pressure on the downstream side, and may be, for example, a pressure increasing valve or a pressure reducing valve. In the present embodiment, as an example, a pressure reducing valve such as a needle valve or a diaphragm valve can be adopted. The pressure adjustment unit 25 can be, for example, accommodated in the housing 10.

The connection portion 8 is an interface such as a joint for connecting the supply source of the oxygen-containing gas. For example, the oxygen cylinder B as a supply source of an oxygen-containing gas is connected to the connection portion 8. The above-described pressure adjustment unit 25 and gas supply unit 21 are connected to the downstream side of the connection portion 8, and the oxygen-containing gas is supplied to the catheter connection portion 1 via the gas supply unit 21. The connection portion 8 is accommodated in the housing 10, for example. An accommodating portion 18 for accommodating the oxygen cylinder B may be provided in the housing 10.

The infusion solution container 7 is a container such as a drip bag that stores an infusion solution. As an example, the infusion solution container 7 is a bag or a cylindrical container, which is liquid-tight, from which the infusion solution does not leak. The infusion solution container 7 can include, on a partition wall portion of the container body, a catheter insertion portion 71 into which the catheter 6 is inserted, and a discharge port 72 for discharging the infusion solution. An infusion solution tube 75 that supplies the infusion solution to a patient is connected to the discharge port 72. In the present embodiment, the catheter insertion portion 71 and the discharge port 72 are one of a plurality of (three or four as an example) insertion ports provided in the rubber plug portion 70 provided at the lower end portion of the infusion solution container 7. As illustrated in FIG. 3, the drip tube 76 is connected to the infusion solution tube 75, and an infusion solution pump 77 and a clamp 78 are provided.

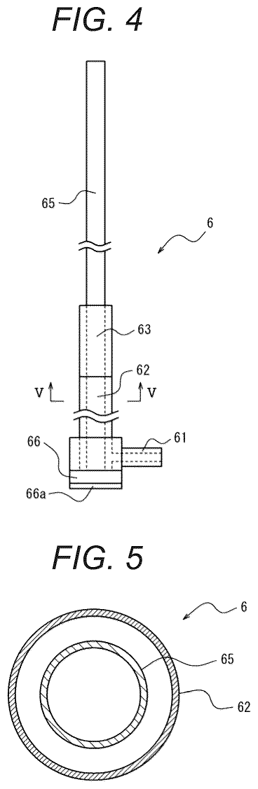

As illustrated in FIGS. 1 and 4, the catheter 6 includes a gas tube connection portion 61 to which a supply tube 16 (see FIG. 1) is connected, a supply pipe 62 to which an oxygen-containing gas is supplied from the gas tube connection portion 61, a gas dispersion mechanism 63 that is provided at an outlet of the supply pipe 62 and exhausts the oxygen-containing gas to the infusion solution container 7, an exhaust pipe 65 that exhausts gas (hereinafter referred to as exhaust gas) from the infusion solution container 7, and an exhaust valve 66 that releases exhaust gas from the exhaust pipe 65 to the outside of the infusion solution container 7. The catheter 6 may be formed in, for example, a linear rod shape.

As illustrated in FIG. 1, the gas tube connection portion 61 is a joint mechanism for connecting the supply tube 16. Any structure may be used for the gas connection portion 61 as long as it is airtight and does not unexpectedly come off.

As illustrated in FIGS. 1 and 4, the supply pipe 62 is a pipe member that forms a supply lumen for supplying the oxygen-containing gas into the infusion solution container 7. The supply pipe 62 is communicably connected to the gas tube connection portion 61, and supplies the oxygen-containing gas supplied from the gas tube connection portion 61 into the infusion solution in the infusion solution container 7 (see FIG. 1) via the gas dispersion mechanism 63 provided at the outlet on the downstream side in the flow of the oxygen-containing gas.

The gas dispersion mechanism 63 may be formed of, for example, a mesh-like member (for example, mesh or sponge), and can supply the oxygen-containing gas supplied from the supply pipe 62 as fine bubbles during infusion, which makes it possible to improve the efficiency of supplying oxygen to the infusion solution, that is, dissolving oxygen in the infusion solution. The supply pipe 62 may be provided with a check valve that prevents backflow of the oxygen-containing gas and the infusion solution.

The exhaust pipe 65 is a pipe member that forms an exhaust lumen for exhausting exhaust gas from the inside of the infusion solution container 7. One end (inlet of the exhaust gas) of the exhaust pipe 65 is opened on the infusion solution container 7 (see FIG. 1), the exhaust gas is taken into the exhaust pipe from the inside of the infusion solution container 7, and the exhaust gas is released to the outside of the infusion solution container 7 through the exhaust valve 66 connected to the other end side. At the inlet of the exhaust pipe 65, a filter such as a net or a porous body that suppresses intrusion of the infusion solution may be provided as necessary.

As the exhaust valve 66, a check valve that prevents backflow of external air into the infusion solution container 7 (see FIG. 1) can be adopted. By preventing backflow of external air into the infusion solution container 7, infection can be prevented. In addition, the exhaust valve 66 may have an adjustment mechanism of flow rate or pressure such as a needle valve structure or a diaphragm valve structure in addition to a one-way valve such as a duckbill valve, and may be capable of adjusting the internal pressure of the infusion solution container 7 or may be an air permeable filter that does not allow liquid to pass, which makes it possible to improve the efficiency of oxygen dissolution in the infusion solution and adjust the oxygen concentration in the infusion solution. A filter 66a may be provided at the outlet portion of the exhaust valve 66 in order to prevent intrusion of bacteria and viruses to prevent contamination of the inside of the infusion solution container 7 from the outside. Specifically, it is preferred that the pore diameter of the filter 66a is selected according to the contaminant to be prevented, and a filter having a pore diameter, for example, from 0.8 μm to 0.1 μm is often used. For example, a filter having a pore diameter of 0.45 μm is often used for the purpose of preventing bacterial contamination.

FIGS. 4 and 5 illustrate a case where the catheter 6 has a double tube structure. In the examples illustrated in FIGS. 4 and 5, a case where the supply pipe 62 is formed as an outer tube and the exhaust pipe 65 is formed as an inner tube is illustrated. In this case, the supply lumen is an inner space of the supply pipe 62 and an outer space of the exhaust pipe 65. In this case, the gas dispersion mechanism 63 may be disposed along the outer peripheral surface of the exhaust pipe 65 at the outlet-side end portion of the supply pipe 62.

As illustrated in FIG. 1, one end of the exhaust pipe 65, that is, the inlet of the exhaust gas in the exhaust pipe 65 is disposed above the gas dispersion mechanism 63 in a state where the catheter 6 is inserted into the infusion solution container 7. In a state where the catheter 6 is inserted into the infusion solution container 7, the inlet of the exhaust gas in the exhaust pipe 65 is disposed so as to be positioned above a liquid level H of the infusion solution. The gas dispersion mechanism 63 is disposed in the infusion solution, that is, below the liquid level H of the infusion solution. The gas tube connection portion 61 and the exhaust valve 66 are disposed outside the infusion solution container 7 in a state where the catheter 6 is inserted into the infusion solution container 7.

The distal end portion of the catheter 6 (the end portion on the side inserted into the infusion solution container 7) is preferably made of a soft material or has a flexible structure in order to avoid breakage (for example, in a case where the infusion solution container 7 is a drip bag, breakage as damage of the bag, or the like) of the infusion solution container 7. For example, the distal end portion of the exhaust pipe 65 may be formed of a rubber-like member, or the distal end portion of the exhaust pipe 65 may have a bellows structure to impart flexibility.

In addition, the distal end portion of the catheter 6 may be provided with, for example, a structure or a mechanism (for example, a structure that opens in a cross shape and a structure or a mechanism that opens at the distal end like a pantograph, which are not illustrated) that opens at the distal end portion. With such a structure or mechanism, the position of the catheter 6 can be fixed in the infusion solution container 7.

As illustrated in FIG. 2, the container pressure sensing unit 31 may be a sensor that senses the internal pressure of the infusion solution container 7 (see FIG. 1), a circuit that senses the internal pressure of the infusion solution container 7 based on a signal output from the sensor, or a functional unit implemented by execution of a program by a CPU or the like. By sensing the internal pressure of the infusion solution container 7, it is possible to prevent the rupture of the infusion solution container 7 and the administration of the infusion solution from the infusion solution container 7 to a patient at an excessive rate and to perform control while ensuring safety. In addition, the oxygen concentration of the infusion solution can be adjusted by sensing the internal pressure of the infusion solution container 7. In addition, due to these actions or effects, the user can easily supply oxygen to the infusion solution and can work quickly (i.e., oxygen can be quickly supplied to the infusion solution).

In the present embodiment, the container pressure sensing unit 31 may include a sensor that senses pressure. For example, the container pressure sensing unit 31 can indirectly sense the internal pressure of the infusion solution container 7 by sensing the pressure of the oxygen-containing gas in the supply tube 16 via the tube 13a connected to the pressure detection port 13 provided in the catheter connection portion 1. FIG. 1 illustrates a case where the tube 13a is connected to the pressure sensor connection portion 13b provided in the housing 10. That is, the container pressure sensing unit 31 is connected to the supply tube 16 via the pressure sensor connection portion 13b.

The oxygen concentration sensing unit 32 illustrated in FIG. 2 may be a sensor that senses the oxygen concentration (for example, oxygen concentration (% O2, g/L), oxygen partial pressure (mmHg, Torr, mbar), oxygen saturation (%), and the like) of the infusion solution, a circuit that senses the oxygen concentration of the infusion solution on the basis of a signal output from the sensor, or a functional unit implemented by execution of a program by a CPU or the like. The oxygen concentration of the infusion solution can be controlled by sensing the oxygen concentration of the infusion solution.

The oxygen concentration sensing unit 32 may sense the oxygen concentration of the infusion solution on the basis of, for example, a signal sent from an oxygen concentration sensor 32a (see FIG. 1) installed in the infusion solution container 7. As an example, the oxygen concentration sensor 32a may be attached to the catheter 6 (see FIG. 1) and installed in the infusion solution container 7 by attaching the catheter 6 to the infusion solution container 7 (see FIG. 1). In this case, the catheter 6 may include the oxygen concentration sensor 32a. The oxygen concentration sensor 32a may be of any type as long as the oxygen concentration sensor 32a can ensure cleanliness.

The temperature sensing unit 33 may be a sensor that senses the temperature of the infusion solution, a circuit that senses the temperature of the infusion solution on the basis of a signal output from the sensor, or a functional unit implemented by execution of a program by a CPU or the like. The oxygen concentration of the infusion solution can be controlled by sensing the oxygen concentration of the infusion solution.

The temperature sensing unit 33 may sense the oxygen concentration of the infusion solution on the basis of, for example, a signal sent from a temperature sensor 33a (see FIG. 1) installed in the infusion solution container 7 (see FIG. 1). As an example, the temperature sensor 33a may be attached to the catheter 6 (see FIG. 1) and installed in the infusion solution container 7 by attaching the catheter 6 to the infusion solution container 7. In this case, the catheter 6 may include the temperature sensor 33a. The temperature sensor 33a may be of any type as long as the temperature sensor 33a can ensure cleanliness.

The remaining amount sensing unit 34 may be a sensor that senses the remaining amount of the infusion solution in the infusion solution container 7, a circuit that senses the temperature of the infusion solution on the basis of a signal output from the sensor, or a functional unit implemented by execution of a program by a CPU or the like. The oxygen concentration of the infusion solution can be appropriately controlled by sensing the remaining amount of the infusion solution.

For example, the remaining amount sensing unit 34 may sense the remaining amount of the infusion solution on the basis of a sensor that senses the weight of the infusion solution container 7 (see FIG. 1) or a signal sent from the sensor. For example, when a supply device such as a drip pump is used for administration of the infusion solution, the remaining amount sensing unit may calculate and sense the remaining amount of the infusion solution on the basis of the operation state of the supply device. As a specific example, for example, the remaining amount of the infusion solution may be obtained by back calculation from the integration of the rotational speed of the drip pump.

The biological information acquisition unit 35 may be a sensor that acquires biological information such as SpO2, a respiratory rate or a respiratory amount, a heart rate, a blood pressure, an electrocardiogram, an electroencephalogram, an inhaled oxygen concentration, a tissue oxygen saturation, an arterial oxygen partial pressure, a mixed venous blood oxygen saturation, a urine oxygen partial pressure, an intravesical pressure, a brain/tissue oxygen saturation, a urine amount, a cardiac output, a body temperature, a sweating state, patient movement, and a patient image, a circuit that senses a temperature of an infusion solution on the basis of a signal output from the sensor, or a functional unit realized by execution of a program by a CPU or the like. The use of the biological information enables appropriate control of the oxygen concentration of the infusion solution based on the condition of the patient. The sensor that acquires the biological information may be any medical device that acquires the biological information of the patient. An example of a sensor for acquiring biological information is an arterial oxygen saturation sensor 35a (see FIG. 3) such as a pulse oximeter for acquiring SpO2 of a patient.

The notification unit 90 illustrated in FIGS. 1 and 2 notifies the user of the operation state of the infusion solution oxygenation device 100 and the notification from the infusion solution oxygenation device 100. In the present embodiment, as illustrated in FIG. 1, the notification unit 90 includes a display unit 91 that performs notification by display, such as a liquid crystal monitor, and a voice output unit 92 that performs notification by voice, such as a speaker.

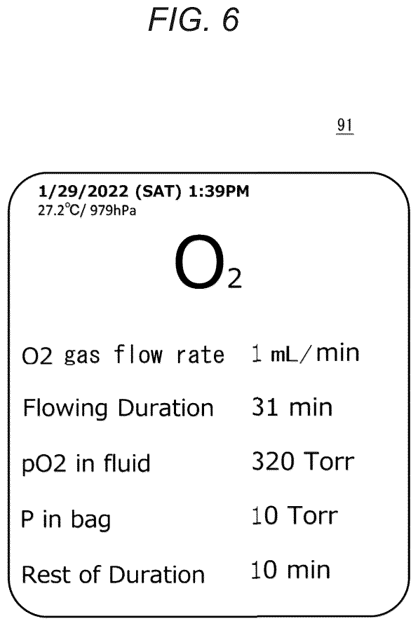

As an example, as illustrated in FIG. 6, the display unit 91 may display information such as a gas type (O2 as an example in FIG. 6), a supply rate of oxygen to the infusion solution (O2 gas flow rate), a time during which the oxygen-containing gas is supplied (Flowing Duration), an internal pressure of the infusion solution container 7 (P in bag), and a remaining scheduled supply time of the oxygen-containing gas (Rest of Duration).

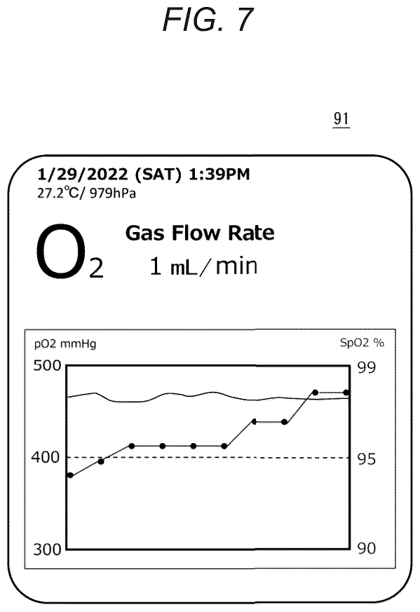

As another example, as illustrated in FIG. 7, information such as a graph of the oxygen partial pressure of the infusion solution (oxygen partial pressure, pO2 in fluid) and a graph of the arterial oxygen saturation (SpO2) may be displayed on the display unit 91 together with the supply rate of the oxygen-containing gas (Gas Flow Rate).

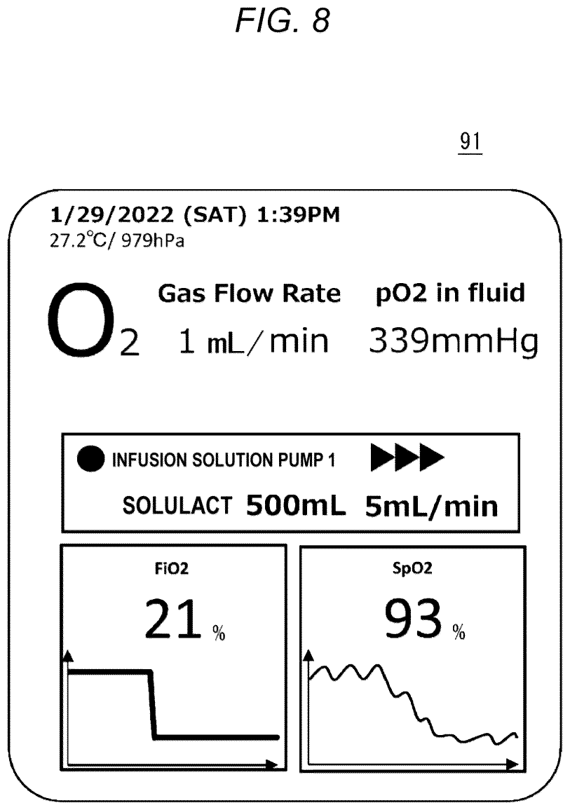

As another example, as illustrated in FIG. 8, the display unit 91 may display information such as the supply rate of the oxygen-containing gas (Gas Flow Rate), the oxygen partial pressure of the infusion solution (pO2 in fluid), the operation state of the infusion solution pump (see FIG. 3) or the administration rate of the infusion solution, the oxygen concentration in the inhaled gas (FiO2, fraction of inspiratory oxygen), and SpO2.

In addition, the display unit 91 may display information related to general information or medical information such as a date, time, patient identification information, and attending physician identification information.

The voice output unit 92 may output the state of the infusion solution oxygenation device 100 by voice. The sound output unit 92 may issue a warning sound at the time of an abnormality of the infusion solution oxygenation system 200.

The input unit 95 illustrated in FIGS. 1 and 2 is an interface that receives an input of an operation command (for example, an instruction to start supplying the oxygen-containing gas) from a user. The input unit 95 may be a connection interface of the input device, a functional unit that receives an input of the operation command via the communication unit W, an input sensor such as a touch panel provided in the display unit 91, or the like. In the present embodiment, as an example, the display unit 91 which is a liquid crystal monitor has a function of a touch panel and also serves as the input unit 95. The display unit 91 may display, as the input unit 95, an image serving as an operation interface for instructing start of supply of the oxygen-containing gas, instructing end of supply, and other setting changes, separately from or simultaneously with the various types of information illustrated in FIGS. 6 to 8.

Hereinafter, a specific operation of the infusion solution oxygenation device 100 will be described.

The control unit C illustrated in FIG. 2 can control the supply of the oxygen-containing gas by the gas supply unit 21 on the basis of the internal pressure of the infusion solution container 7 (see FIG. 1) sensed by the container pressure sensing unit 31. The control unit C controls the supply of the oxygen-containing gas by the gas supply unit 21 based on the internal pressure of the infusion solution container 7 so as not to rupture the infusion solution container 7 or administer the infusion solution from the infusion solution container 7 to the patient at an excessive rate. For example, when the internal pressure of the infusion solution container 7 becomes too high, the control unit C reduces the supply rate of the oxygen-containing gas to the gas supply unit 21, and continues the supply of oxygen to the patient while preventing the rupture of the infusion solution container 7 and the administration of the infusion solution from the infusion solution container 7 to the patient at an excessive rate.

The control unit C may control the supply of the oxygen-containing gas by the gas supply unit 21 on the basis of the oxygen concentration of the infusion solution sensed by the oxygen concentration sensing unit 32. For example, when the oxygen concentration of the infusion solution is lower than a predetermined target value, the control unit C may increase the supply rate of the oxygen-containing gas by the gas supply unit 21. As a result, it is possible to promote the dissolution of oxygen in the infusion solution to increase the oxygen concentration of the infusion solution and to avoid the shortage of oxygen supplied to the patient. For example, when the oxygen concentration of the infusion solution is higher than a predetermined target value, the control unit C may lower the supply rate of the oxygen-containing gas by the gas supply unit 21. As a result, the oxygen concentration of the infusion solution can be reduced. As a result, for example, it is possible to avoid an accident in which air bubbles are generated in the body (blood vessel) of the patient and an air embolus occurs. The control unit C may decrease the oxygen concentration of the supply gas instead of the method of decreasing the supply rate of the oxygen-containing gas by the gas supply unit 21. For example, it can be realized by mixing nitrogen, argon, carbon dioxide, nitrogen monoxide, nitrous oxide gas, atmospheric air, or the like with the supply gas and controlling the mixing ratio.

The control unit C may adjust the internal pressure of the infusion solution container 7 (see FIG. 1) by the pressure adjustment unit 25 on the basis of the oxygen concentration of the infusion solution sensed by the oxygen concentration sensing unit 32. For example, when the oxygen concentration of the infusion solution is lower than a predetermined target value, the control unit C may increase the internal pressure of the infusion solution container 7 by the pressure adjustment unit 25. As a result, it is possible to promote the dissolution of oxygen in the infusion solution to increase the oxygen concentration of the infusion solution and to avoid the shortage of oxygen supplied to the patient. For example, when the oxygen concentration of the infusion solution is higher than a predetermined target value, the control unit C may lower (reduce) the internal pressure of the infusion solution container 7 by the pressure adjustment unit 25. As a result, the oxygen concentration of the infusion solution can be reduced. As a result, for example, it is possible to avoid an accident in which air bubbles are generated in the body (blood vessel) of the patient and an air embolus occurs.

The control unit C may control the supply of the oxygen-containing gas by the gas supply unit 21 on the basis of the temperature of the infusion solution sensed by the temperature sensing unit 33. For example, when the temperature of the infusion solution is higher than the reference temperature, the control unit C may increase the supply rate of the oxygen-containing gas by the gas supply unit 21. As a result, it is possible to avoid a decrease in the oxygen concentration of the infusion solution due to an increase in the temperature of the infusion solution and to avoid a shortage of oxygen supplied to the patient. When the temperature of the infusion solution is lower than the reference temperature, the control unit C may decrease the supply rate of the oxygen-containing gas by the gas supply unit 21, which makes it possible to avoid an excessive increase in the oxygen concentration of the infusion solution accompanying a decrease in the temperature of the infusion solution.

The control unit C may control the supply of the oxygen-containing gas by the gas supply unit 21 on the basis of the remaining amount of the infusion solution sensed by the remaining amount sensing unit 34. As a result, it is possible to prevent consumption of the oxygen-containing gas that does not contribute to the administration to the patient, that is, unnecessary consumption.

The control unit C may control the supply of the oxygen-containing gas by the gas supply unit 21 on the basis of the biological information acquired by the biological information acquisition unit 35. In addition, the control unit C may adjust the internal pressure of the infusion solution container 7 by the pressure adjustment unit 25 on the basis of the biological information acquired by the biological information acquisition unit 35.

For example, in a case where the biological information includes information indicating lack of oxygen in the patient, the control unit C may control the gas supply unit 21 and the pressure adjustment unit 25 so that the oxygen concentration of the infusion solution increases. These controls are as described above, and the oxygen concentration of the infusion solution can be increased by the control unit C increasing the supply amount of the oxygen supply gas by the gas supply unit 21. The control unit C can increase the oxygen concentration of the infusion solution by increasing the internal pressure of the infusion solution container 7 (see FIG. 1) by the pressure adjustment unit 25.

Note that the case where the biological information includes information indicating lack of oxygen in the patient includes, for example, a case where the value of SpO2 is lower than a predetermined value (for example, 90), a case where the value of SpO2 does not show a tendency to increase even after a predetermined period elapses after the supply of the infusion solution is started, and a case where the value of SpO2 shows a tendency to decrease.

Note that, according to the condition of the patient, for example in a case where the biological information does not include information indicating lack of oxygen in the patient, the control unit C may control the gas supply unit 21 and the pressure adjustment unit 25 so that the oxygen concentration of the infusion solution decreases. These controls are as described above, and the oxygen concentration of the infusion solution can be decreased by the control unit C decreasing the supply amount of the oxygen supply gas by the gas supply unit 21. The control unit C can decrease the oxygen concentration of the infusion solution by decreasing the internal pressure of the infusion solution container 7 (see FIG. 1) by the pressure adjustment unit 25. In addition, the infusion may be stopped in conjunction with the infusion solution pump (see FIG. 3) or the like.

Note that the case where the biological information does not include information indicating lack of oxygen in the patient can be, for example, a case where the value of SpO2 is a predetermined value (for example, 90) or more.

Hereinafter, a method of using the infusion solution oxygenation device 100 will be described.

When the infusion solution oxygenation device 100 is used, it is assumed that an infusion solution is administered to a patient. The infusion solution may be an infusion solution containing a drug used for treatment of the patient, or may be a saline solution or the like when only the purpose is to supply oxygen. It is preferred to select an infusion solution containing water as a component, in which oxygen can be dissolved. When a drug is contained in the infusion solution and the contact of the drug with oxygen is to be avoided, the infusion solution oxygenation device 100 should not be used with such an infusion solution.

When using the infusion solution oxygenation device 100, as illustrated in FIG. 1, first, the catheter 6 is attached to the infusion solution container 7. Next, supply of the oxygen-containing gas from the infusion solution oxygenation device 100 to the infusion solution container 7 is started.

For example, after the supply of the oxygen-containing gas to the infusion solution container 7 is started, the administration of the infusion solution to the patient may be started. The supply of the infusion solution may be started after the oxygen concentration of the infusion solution reaches a predetermined value (target value necessary for treatment) according to the condition of the patient. It should be noted that the infusion solution is administered to the patient in accordance with common knowledge of ordinary medical practice. When the administration of the infusion solution is prioritized over the administration of oxygen, the administration of the infusion solution to the patient may be started before the supply of the oxygen-containing gas to the infusion solution container 7 is started.

After the start of infusion, attention is paid to the notification from the notification unit 90, and treatment is performed on the patient as necessary.

Hereinafter, medical effects of using the infusion solution oxygenation device 100 will be described on the basis of animal experiments.

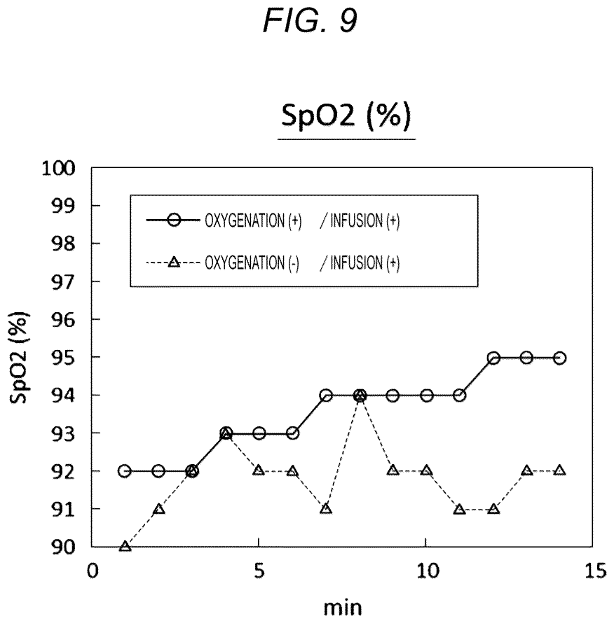

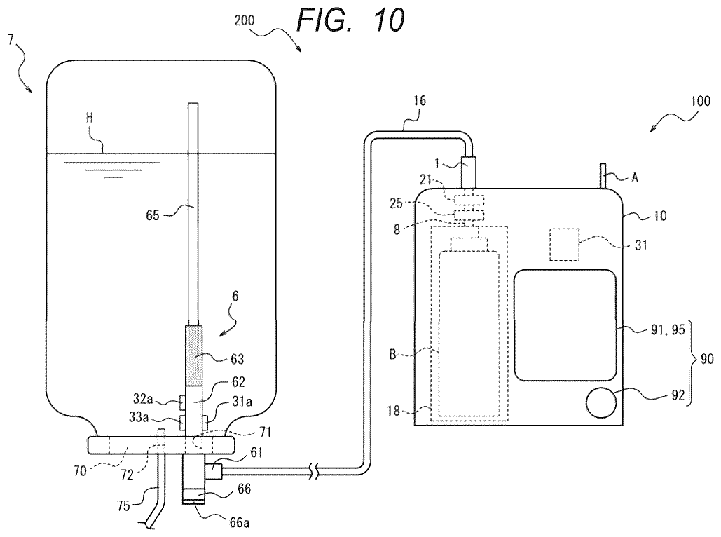

FIG. 9 shows the results of animal experiments using a pig as a test animal. In this animal experiment, first, FiO2 (oxygen concentration in the inhaled gas) of the pig under anesthesia was controlled to about 21% of the atmospheric level, and SpO2 of the pig was lowered to 90. Thereafter, the SpO2 transition in the pig was observed between when the oxygenated infusion solution was administered (see the graph drawn with white circles and a solid line in FIG. 9) and when the infusion solution was not administered (see the graph drawn with white triangles and a broken line in FIG. 9). The observation period was set to 15 minutes. Ringer's lactate was used as the infusion solution. As the experimental results, as shown in FIG. 9, a significant increase in SpO2 could be observed when the oxygenated infusion solution was administered. That is, it was shown that oxygen supply to the test animal by the oxygenated infusion solution was effective.

### Second Embodiment

The second embodiment is different from the first embodiment in the mode of sensing of the internal pressure of the infusion solution container 7 by the container pressure sensing unit 31, and the other configurations are the same. Hereinafter, differences from the first embodiment will be described, and description of common parts will be omitted.

As illustrated in FIG. 10, in the present embodiment, the container pressure sensing unit 31 does not need to include a sensor that senses pressure. In the present embodiment, the container pressure sensing unit 31 may sense the internal pressure of the infusion solution container 7 on the basis of a signal transmitted from the pressure sensor 31a installed in the infusion solution container 7. As an example, the pressure sensor 31a may be attached to the catheter 6 and installed in the infusion solution container 7 by attaching the catheter 6 to the infusion solution container 7. In this case, the catheter 6 may include the pressure sensor 31a. The pressure sensor 31a may be of any type as long as the pressure sensor 31a can ensure cleanliness. In this case, the container pressure sensing unit 31 can directly sense the internal pressure of the infusion solution container 7.

### Third Embodiment

The third embodiment is different from the second embodiment in that the exhaust valve 66 is not provided in the catheter 6, the exhaust from the exhaust pipe 65 is performed via the pressure adjusting exhaust valve 67 (another example of the pressure adjustment unit) provided in the infusion solution oxygenation device 100, and the control of the internal pressure of the infusion solution container 7 by the infusion solution oxygenation device 100 can be performed more precisely, and the others are the same. Hereinafter, differences from the second embodiment will be described, and description of common parts will be omitted.

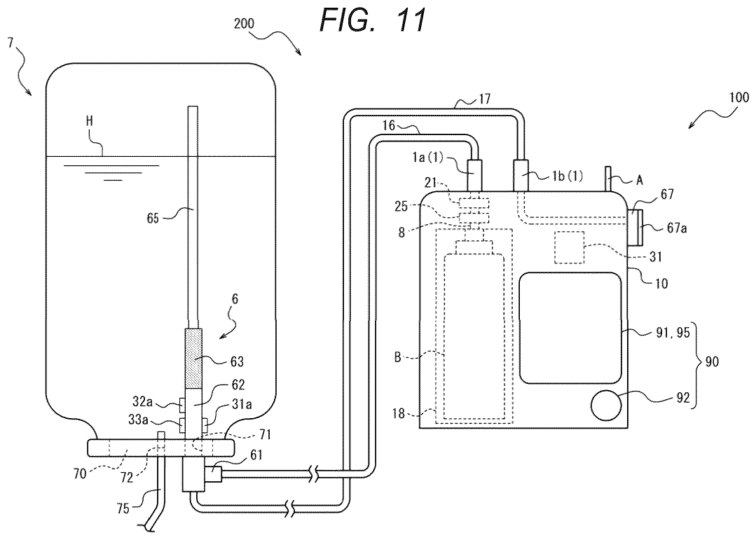

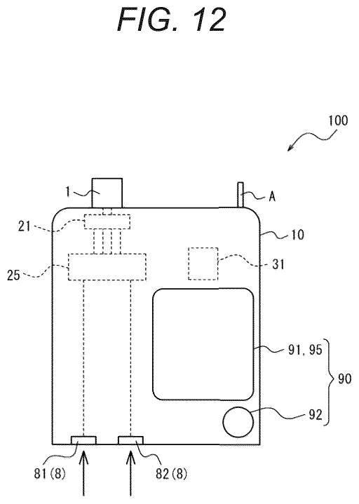

As illustrated in FIG. 11, in the present embodiment, the pressure adjusting exhaust valve 67 is provided in the infusion solution oxygenation device 100. For example, the pressure adjusting exhaust valve 67 may be fixed to the housing 10. In addition to the supply tube 16, an exhaust tube 17 is connected to the catheter 6.

An end portion of the supply tube 16 on the side opposite to the side connected to the catheter 6 is connected to the air supply unit 1a of the catheter connection portion 1. Note that the air supply unit 1a is supplied with the oxygen-containing gas from the gas supply unit 21 (see FIG. 2), and supplies the oxygen-containing gas to the supply tube 16.

One end portion of the exhaust tube 17 is connected to the exhaust pipe 65 (see FIG. 4) of the catheter 6, and the other end portion is connected to the exhaust unit 1b of the catheter connection portion 1. The exhaust unit 1b is communicably connected to the pressure adjusting exhaust valve 67 inside the housing 10, and can discharge the exhaust gas from the exhaust pipe 65 to the outside via the pressure adjusting exhaust valve 67.

In the present embodiment, the pressure adjusting exhaust valve 67 may include an actuator, and may adjust the flow rate of the exhaust gas from the exhaust pipe 65 and the internal pressure of the infusion solution container 7 on the basis of the operation command of the control unit C. The pressure adjusting exhaust valve 67 may be an aperture adjusting valve capable of adjusting the flow rate of the exhaust gas from the exhaust pipe 65 and the internal pressure of the infusion solution container 7 by adjusting the aperture, and as an example, a needle valve or a diaphragm valve may be employed. As a result, the control unit C can adjust the internal pressure of the infusion solution container 7 by controlling the pressure adjusting exhaust valve 67, adjust the efficiency of dissolving oxygen in the infusion solution, and adjust the oxygen concentration in the infusion solution.

Note that a filter 67a may be provided at the outlet portion of the pressure adjusting exhaust valve 67 to prevent contamination of the inside of the infusion solution container 7 from the outside.

### Description of Modifications

### First Modification

In the above embodiment, the case where the supply source of the oxygen-containing gas (for example, the oxygen cylinder B) is connected to the connection portion 8 has been described. However, the component connected to the connection portion 8 is not limited to the supply source of the oxygen-containing gas. For example, as shown in FIG. 12, the connection portion 8 may include a first connection portion 81 connected to a supply source of a first gas containing oxygen gas, and a second connection portion 82 connected to a supply source of a second gas different from the first gas.

The connection portion 8, that is, the first connection portion 81 or the second connection portion 82 is not limited to a so-called gas cylinder, and the first connection portion 81 or the second connection portion 82 may be connected to, for example, an oxygen-containing gas supply pipe or a pipe for supplying the second gas in a hospital. One or more second connection portions 82 may be provided.

The second gas may contain nitrogen, argon, nitrous oxide gas, or carbon dioxide. One or more second gases may be used.

In this case, the gas supply unit 21 can mix the first gas and the second gas at a ratio based on a command from the control unit C (see FIG. 2) to prepare the oxygen-containing gas, and supply the oxygen-containing gas to the catheter 6. As a result, it is possible to prepare an infusion solution with oxygen and other gases dissolved so as to be in a state suited for the condition of the patient. For example, the control unit C may instruct the gas supply unit 21 to prepare the oxygen-containing gas by mixing the first gas and the second gas at an appropriate ratio on the basis of the biological information so that the balance of partial pressures of oxygen, nitrogen, argon, or carbon dioxide dissolved in the infusion solution becomes a state suited for the condition of the patient.

### Second Modification

In the above embodiment, it has been described that the control unit C may control the supply of the oxygen-containing gas by the gas supply unit 21 on the basis of the temperature of the infusion solution sensed by the temperature sensing unit 33. The control unit C may issue a command to the external device to adjust the temperature of the infusion solution, together with the supply control of the oxygen-containing gas by the gas supply unit 21 based on the temperature of the infusion solution sensed by the temperature sensing unit 33, or separately from the supply control of the oxygen-containing gas by the gas supply unit 21 based on the temperature of the infusion solution sensed by the temperature sensing unit 33.

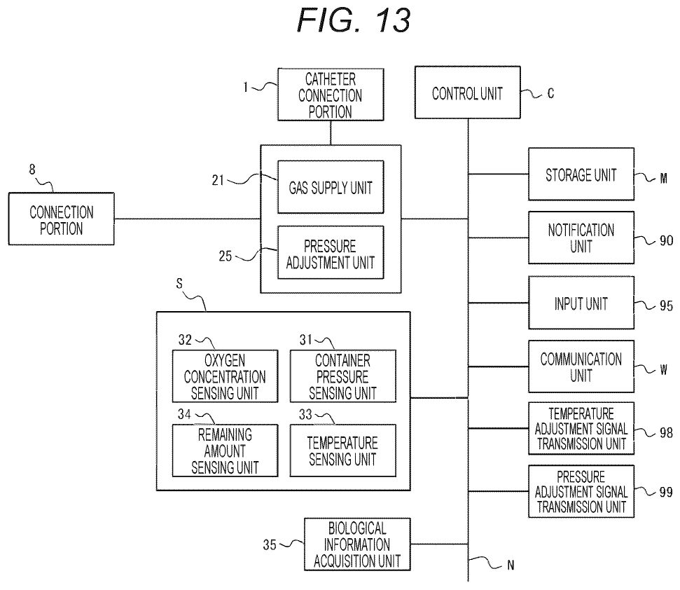

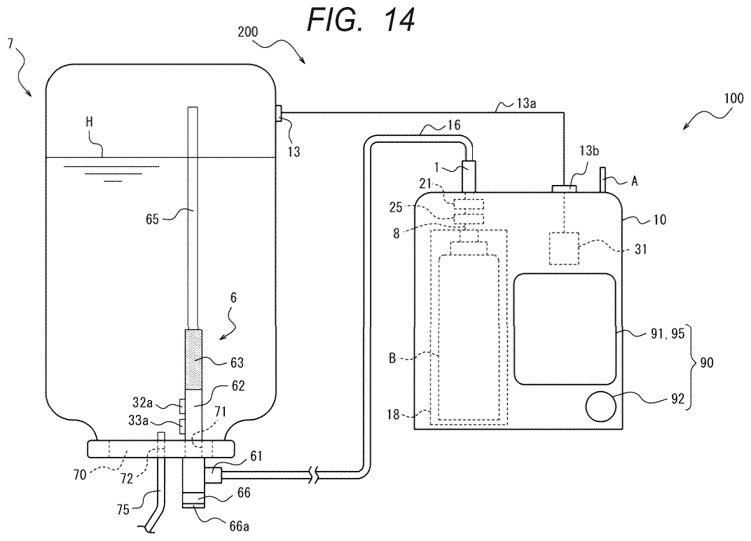

For example, as illustrated in FIG. 13, the infusion solution oxygenation device 100 may include a temperature adjustment signal transmission unit 98 that transmits a temperature adjustment signal including a command to adjust the temperature of the infusion to the external device.

The temperature adjustment signal transmission unit 98 transmits a temperature adjustment signal including a command for adjusting the temperature of the infusion solution to the external device on the basis of a command from the control unit C. In this case, the control unit C may cause the temperature adjustment signal transmission unit 98 to transmit the temperature adjustment signal on the basis of the temperature of the infusion solution sensed by the temperature sensing unit 33 or the oxygen concentration of the infusion solution sensed by the oxygen concentration sensing unit 32.

The control unit C may cause the temperature adjustment signal transmission unit 98 to transmit a temperature adjustment signal so that the temperature of the infusion solution becomes a predetermined target value. As a result, the state of the infusion solution is stabilized, and for example, the oxygen concentration of the infusion solution can be stabilized, whereby the patient can be safely treated.

When the oxygen concentration of the infusion solution sensed by the oxygen concentration sensing unit 32 is high, the control unit C may cause the temperature adjustment signal transmission unit 98 to transmit a temperature adjustment signal including a command to increase the temperature of the infusion solution. As a result, the temperature of the infusion solution increases, and the oxygen concentration of the infusion solution can be reduced. When the oxygen concentration of the infusion solution sensed by the oxygen concentration sensing unit 32 is low, the control unit C may cause the temperature adjustment signal transmission unit 98 to transmit a temperature adjustment signal including a command to lower the temperature of the infusion solution. As a result, the temperature of the infusion solution decreases, and the oxygen concentration of the infusion solution can be increased.

The temperature adjustment signal transmission unit 98 may be a functional unit realized by executing a program by an electric circuit, a CPU, or the like that transmits a signal including a command to adjust the temperature of the infusion solution. The signal including the command to adjust the temperature of the infusion solution may be transmitted to the external device via the communication unit W, or the temperature adjustment signal transmission unit 98 may have a connector unit and transmit the signal by wired connection with the external device.

An example of the external device capable of adjusting the temperature of the infusion solution is a temperature control device (for example, an electric heater or a cooler) that can be attached to the infusion solution container 7 (see FIG. 1). As a result, the temperature of the infusion solution can be increased or decreased. In a case where it is sufficient to only increase the temperature of the infusion solution, an external device that heats the infusion solution tube 75 and the drip tube 76 (see FIG. 3) may be used. In the case of heating the infusion solution tube 75, it is preferable to heat the infusion solution tube 75 on the infusion solution container 7 side rather than on the patient side of the drip tube 76. As a result, air bubbles generated by heating of the infusion solution (extra oxygen removed from the infusion solution by raising the temperature of the infusion solution) can be removed (separated from the infusion solution) in the drip tube 76. It is usually more desirable to heat the infusion solution by heating the drip tube 76 than to heat the infusion solution tube 75. This is because the drip tube 76 has a predetermined container volume and has a long retention time of the infusion solution, so that the temperature can be reliably raised to a desired temperature, which makes it possible to more reliably control the oxygen concentration of the infusion solution.

### Third Modification

In the above embodiment, it has been described that the control unit C can control the supply of the oxygen-containing gas by the gas supply unit 21 on the basis of the internal pressure of the infusion solution container 7 sensed by the container pressure sensing unit 31.

Together with the supply control of the oxygen-containing gas by the gas supply unit 21 based on the internal pressure of the infusion solution container 7 sensed by the container pressure sensing unit 31 or separately from the supply control of the oxygen-containing gas by the gas supply unit 21 based on the internal pressure of the infusion solution container 7 sensed by the container pressure sensing unit 31, the control unit C may issue a command for adjusting the internal pressure of the infusion solution container 7 to the external device.

For example, as illustrated in FIG. 13, the infusion solution oxygenation device 100 may include a pressure adjustment signal transmission unit 99 that transmits a pressure adjustment signal including a command to adjust the internal pressure of the infusion solution container 7 to the external device.

The pressure adjustment signal transmission unit 99 transmits a pressure adjusting signal including a command to adjust the internal pressure of the infusion solution container 7 (see FIG. 1) to the external device on the basis of a command from the control unit C. In this case, the control unit C causes the pressure adjustment signal transmission unit 99 to transmit the pressure adjusting signal on the basis of the internal pressure of the infusion solution container 7 sensed by the container pressure sensing unit 31 or the oxygen concentration of the infusion solution sensed by the oxygen concentration sensing unit 32.

The control unit C may cause the pressure adjustment signal transmission unit 99 to transmit a temperature adjustment signal so that the internal pressure of the infusion solution container 7 becomes a predetermined target value. As a result, the internal pressure of the infusion solution container 7 (see FIG. 1) is stabilized, and for example, the oxygen concentration of the infusion solution can be stabilized, and the patient can be safely treated.

When the oxygen concentration of the infusion solution sensed by the oxygen concentration sensing unit 32 is high, the control unit C may cause the pressure adjustment signal transmission unit 99 to transmit a pressure adjusting signal including a command to lower the internal pressure of the infusion solution container 7 (see FIG. 1). As a result, the internal pressure of the infusion solution container 7 decreases, and the oxygen concentration of the infusion solution can be reduced. When the oxygen concentration of the infusion solution sensed by the oxygen concentration sensing unit 32 is low, the control unit C may cause the pressure adjustment signal transmission unit 99 to transmit a pressure adjusting signal including a command to increase the internal pressure of the infusion solution container 7. As a result, the internal pressure of the infusion solution container 7 increases, and the oxygen concentration of the infusion solution can be increased.

The pressure adjustment signal transmission unit 99 may be a functional unit realized by executing a program by an electric circuit, a CPU, or the like that transmits a pressure adjusting signal including a command to adjust the internal pressure of the infusion solution container 7 (see FIG. 1). The signal including the command to adjust the internal pressure of the infusion solution container 7 may be sent to an external device via the communication unit W, or the pressure adjustment signal transmission unit 99 may have a connector unit and transmit the signal by wired connection with the external device.

An example of an external device capable of adjusting the internal pressure of the infusion solution container 7 illustrated in FIG. 1 is a pressurizing device that compresses and pressurizes the infusion solution container 7, or a decompression device that has a space for accommodating the infusion solution container 7 and decompresses the interior of the infusion solution container 7 by decompressing the space.

Note that, in a case where the oxygen concentration of the infusion solution is likely to increase, or in a case where it is desired to rapidly decrease the oxygen concentration of the infusion solution when the oxygen concentration of the infusion solution increases, it is preferable to use a decompression device capable of decompressing the inside of the infusion solution container 7 to a pressure lower than the atmospheric pressure. In the case of using such a decompression device, when the oxygen concentration of the infusion solution sensed by the oxygen concentration sensing unit 32 is higher than a predetermined target value, the control unit C illustrated in FIG. 2 may cause the pressure adjustment signal transmission unit 99 to transmit a temperature adjustment signal including a command to lower the internal pressure of the infusion solution container 7 (see FIG. 1) to be lower than the atmospheric pressure. As a result, the oxygen concentration of the infusion solution can be controlled to be low, and an increase in the oxygen concentration of the infusion solution can be prevented.

As described above, it is possible to provide an infusion solution oxygenation device and an infusion solution oxygenation system capable of supplying oxygen to an infusion solution safely, simply, and quickly.

### Other Embodiments

(1) In the above embodiment, the case where the oxygen concentration sensor 32a is installed in the infusion solution container 7 has been described, but the oxygen concentration sensor 32a may be provided in the infusion solution tube 75 or the drip tube 76 to sense the oxygen concentration of the infusion solution in the infusion solution tube 75 or the drip tube 76. As a result, the control unit C can perform necessary control on the basis of the oxygen concentration of the infusion solution immediately before being administered to the patient.

(2) In the above embodiment, the case where the temperature sensor 33a is installed in the infusion solution container 7 has been described, but the temperature sensor 33a may be provided in the infusion solution tube 75 or the drip tube 76 to sense the temperature of the infusion solution in the infusion solution tube 75 or the drip tube 76. As a result, the control unit C can perform necessary control on the basis of the temperature of the infusion solution immediately before being administered to the patient.

(3) In the above embodiment, the case where the exhaust gas is exhausted to the outside via the exhaust valve 66 or the pressure adjusting exhaust valve 67 has been described. However, together with the exhaust valve 66 or the pressure adjusting exhaust valve 67, or instead of the exhaust valve 66 or the pressure adjusting exhaust valve 67, a degassing valve may be provided in the infusion solution container 7, and the exhaust gas may be exhausted from the degassing valve. In this case, the exhaust pipe 65, the exhaust valve 66, and the pressure adjusting exhaust valve 67 may be omitted.

(4) In the above embodiment, the case where the display unit 91 which is a liquid crystal monitor has the function of a touch panel and also serves as the input unit 95 has been described. As another example of the input unit 95, the infusion solution oxygenation device 100 may include a voice input unit such as a microphone. That is, the input unit 95 may receive an instruction input by a user's voice.

(5) In the above embodiment, the case where the catheter 6 has a double tube structure has been described as an example. However, the catheter 6 is not limited to the double tube structure. For example, the catheter 6 may be formed by adjacently integrating the supply pipe 62 and the exhaust pipe 65. Alternatively, the supply pipe 62 and the exhaust pipe 65 may be inserted as completely separate components. In addition, the supply pipe 62 and the exhaust pipe 65 may be separated from each other, and may be bundled and integrated at their root portions (for example, a portion where the gas tube connection portion 61 is provided).

(6) In the first embodiment, the case where the container pressure sensing unit 31 senses the pressure of the oxygen-containing gas in the supply tube 16 via the tube 13a connected to the pressure detection port 13 provided in the catheter connection portion 1, thereby indirectly sensing the internal pressure of the infusion solution container 7 has been described as an example. However, as illustrated in FIG. 14, the pressure detection port 13 may be provided in the infusion solution container 7 so that the container pressure sensing unit 31 can directly sense the internal pressure of the infusion solution container 7.

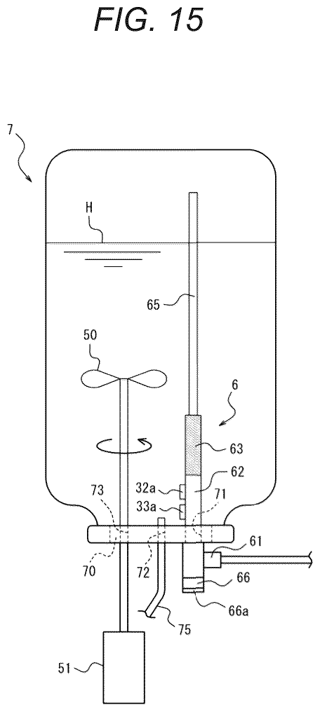

(7) In the above embodiment, the aspect in which the catheter 6 releases and supplies the oxygen-containing gas in the infusion solution in the infusion solution container 7 to dissolve oxygen in the infusion solution has been described. In the infusion solution container 7, the infusion solution may be stirred to promote dissolution of the oxygen-containing gas released by the catheter 6 in the infusion solution. In this case, as illustrated in FIG. 15, the infusion solution may be stirred by the stirrer 5 including the stirring paddle 50 that stirs the inside of the infusion solution container 7 and the drive unit 51 such as a motor that rotates the stirring paddle 50. The stirring paddle 50 may be inserted into the infusion solution container 7 from the stirrer insertion port 73, which is one of the insertion ports of the rubber plug portion 70, which makes it possible to quickly dissolve oxygen and ensure the speed at which the oxygen concentration reaches a predetermined oxygen concentration and the uniformity of the solution. The shape of the stirring paddle 50 is not limited to the paddle shape, and is only required to be able to stir the solution.

(8) In the above embodiment, for example, as illustrated in FIG. 1, the case where the infusion solution container 7 includes the catheter insertion portion 71 into which the catheter 6 is inserted and the discharge port 72 that discharges the infusion solution has been described as an example. In addition, the case where the catheter 6 has a double pipe structure, the supply pipe 62 is an outer tube, and the exhaust pipe 65 is formed as an inner tube has been described. However, the catheter insertion portion 71 and the discharge port 72 for discharging an infusion solution should not be necessarily separated in the infusion solution container 7. For example, the catheter insertion portion 71 may also serve as the discharge port 72. In this case, in addition to the supply pipe 62 and the exhaust pipe 65, a discharge pipe substituting the discharge port 72 may be further provided in the catheter 6, and the infusion solution may be discharged from the discharge pipe.

Note that, the configuration disclosed in the above-described embodiments (including another embodiment, the same applies hereinafter) can be applied in combination with the configuration disclosed in other embodiments as long as there is no contradiction, and the embodiment disclosed in the present specification is an example, and the embodiment of the present disclosure is not limited thereto, and can be appropriately modified without departing from the object of the present disclosure.

The detailed description above describes embodiments of an infusion solution oxygenation device and an infusion solution oxygenation system. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An infusion solution oxygenation device comprising:
a catheter connection portion connected to a catheter, the catheter configured to supply an oxygen-containing gas to an infusion solution in an infusion solution container;
a gas supply unit configured to supply the oxygen-containing gas to the catheter connection portion;
a container pressure sensing unit configured to sense an internal pressure of the infusion solution container; and
wherein the gas supply unit is configured to control supply of the oxygen-containing gas based on the internal pressure sensed by the container pressure sensing unit.

2. The infusion solution oxygenation device according to claim 1, wherein the catheter connection portion comprises:
an air supply unit configured to supply the oxygen-containing gas into the infusion solution container; and
an exhaust unit configured to exhaust gas from the inside of the infusion solution container.

3. The infusion solution oxygenation device according to claim 1, further comprising:
an oxygen concentration sensing unit configured to sense an oxygen concentration of the infusion solution; and
wherein the gas supply unit is configured to control the supply of the oxygen-containing gas based on the oxygen concentration sensed by the oxygen concentration sensing unit.

4. The infusion solution oxygenation device according to claim 3, further comprising:
a pressure adjustment unit configured to adjust an internal pressure of the infusion solution container; and
wherein the pressure adjustment unit is configured to adjust the internal pressure based on the oxygen concentration sensed by the oxygen concentration sensing unit.

5. The infusion solution oxygenation device according to claim 1, further comprising:
a biological information acquisition unit configured to acquire biological information of a patient; and
wherein the gas supply unit is configured to control the supply of the oxygen-containing gas based on the biological information acquired by the biological information acquisition unit.

6. The infusion solution oxygenation device according to claim 5, wherein the biological information includes a blood oxygen saturation, an inhaled oxygen concentration, a blood pressure, a heart rate, a respiratory rate, a tissue oxygen saturation, an arterial oxygen partial pressure, a mixed venous blood oxygen saturation, a urine oxygen partial pressure, an intravesical pressure, a brain/tissue oxygen saturation, a urine amount, a body temperature, or a cardiac output.

7. The infusion solution oxygenation device according to claim 1, further comprising:
a remaining amount sensing unit configured to sense a remaining amount of the infusion solution in the infusion solution container; and
wherein the gas supply unit is configured to control the supply of the oxygen-containing gas based on the remaining amount of the infusion solution acquired by the remaining amount sensing unit.

8. The infusion solution oxygenation device according to claim 1, further comprising:
a temperature sensing unit configured to sense a temperature of the infusion solution; and
wherein the gas supply unit is configured to control the supply of the oxygen-containing gas based on the temperature sensed by the temperature sensing unit.

9. The infusion solution oxygenation device according to claim 1, further comprising:
an oxygen concentration sensing unit configured to sense an oxygen concentration of the infusion solution;
a temperature sensing unit configured to sense a temperature of the infusion solution;
a temperature adjustment signal transmission unit configured to transmit a temperature adjustment signal for adjusting the temperature of the infusion solution; and
wherein the temperature adjustment signal transmission unit is configured to transmit the temperature adjustment signal based on the temperature sensed by the temperature sensing unit or the oxygen concentration sensed by the oxygen concentration sensing unit.

10. The infusion solution oxygenation device according to claim 1, further comprising:
an oxygen concentration sensing unit configured to sense an oxygen concentration of the infusion solution;
a pressure adjustment signal transmission unit configured to transmit a pressure adjustment signal for adjusting an internal pressure of the infusion solution container; and
wherein the pressure adjustment signal transmission unit is configured to transmit the pressure adjustment signal based on the internal pressure sensed by the container pressure sensing unit or the oxygen concentration sensed by the oxygen concentration sensing unit.

11. The infusion solution oxygenation device according to claim 1, further comprising:
a first connection portion connected to a supply source of a first gas containing an oxygen gas;
a second connection portion connected to a supply source of a second gas different from the first gas;
wherein the second gas contains nitrogen, argon, nitrogen monoxide, nitrous oxide gas, or carbon dioxide; and
the gas supply unit is configured to prepare the oxygen-containing gas by mixing the first gas and the second gas.

12. An infusion solution oxygenation system comprising:
a catheter configured to supply an oxygen-containing gas to an infusion solution in an infusion solution container;
a gas supply unit configured to supply the oxygen-containing gas to the catheter;
a container pressure sensing unit configured to sense an internal pressure of the infusion solution container; and
wherein the gas supply unit is configured to control supply of the oxygen-containing gas based on the internal pressure sensed by the container pressure sensing unit.

13. The infusion solution oxygenation system according to claim 12, wherein the catheter comprises:
a supply lumen configured to supply the oxygen-containing gas into the infusion solution container; and
an exhaust lumen configured to exhaust gas from the inside of the infusion solution container.

14. A method for supplying to an infusion solution, the method comprising:
supplying an oxygen-containing gas to an infusion solution in an infusion solution container via a catheter, the catheter being connected to a catheter connection portion;
supplying the oxygen-containing gas to the catheter connection portion via a gas supply unit;
sensing an internal pressure of the infusion solution container with a container pressure sensing unit; and
controlling the supply of the oxygen-containing gas by the gas supply unit based on the internal pressure sensed by the container pressure sensing unit.

15. The method according to claim 14, further comprising:
supplying the oxygen-containing gas into the infusion solution container with an air supply unit; and
exhausting gas from the inside of the infusion solution container with an exhaust unit.

16. The method according to claim 14, further comprising:
sensing an oxygen concentration of the infusion solution with an oxygen concentration sensing unit; and
controlling the supply of the oxygen-containing gas with the gas supply unit based on the oxygen concentration sensed by the oxygen concentration sensing unit.

17. The method according to claim 16, further comprising:
adjusting an internal pressure of the infusion solution container with a pressure adjustment unit; and
adjusting the internal pressure with the pressure adjustment unit based on the oxygen concentration sensed by the oxygen concentration sensing unit.

18. The method according to claim 14, further comprising:
acquiring biological information of a patient with a biological information acquisition unit; and
controlling the supply of the oxygen-containing gas with the gas supply unit based on the biological information acquired by the biological information acquisition unit.

19. The method according to claim 18, wherein the biological information includes a blood oxygen saturation, an inhaled oxygen concentration, a blood pressure, a heart rate, a respiratory rate, a tissue oxygen saturation, an arterial oxygen partial pressure, a mixed venous blood oxygen saturation, a urine oxygen partial pressure, an intravesical pressure, a brain/tissue oxygen saturation, a urine amount, a body temperature, or a cardiac output.

20. The method according to claim 14, further comprising one or more of:
sensing a remaining amount of the infusion solution in the infusion solution container with a remaining amount sensing unit, and controlling the supply of the oxygen-containing gas with the gas supply unit based on the remaining amount of the infusion solution acquired by the remaining amount sensing unit;
sensing a temperature of the infusion solution with a temperature sensing unit, and controlling the supply of the oxygen-containing gas with the gas supply unit based on the temperature sensed by the temperature sensing unit;
sensing an oxygen concentration of the infusion solution with an oxygen concentration sensing unit, transmitting a temperature adjustment signal for adjusting the temperature of the infusion solution with a temperature adjustment signal transmission unit, and transmitting the temperature adjustment signal based on the temperature sensed by the temperature sensing unit or the oxygen concentration sensed by the oxygen concentration sensing unit; and
sensing the oxygen concentration of the infusion solution with the oxygen concentration sensing unit, transmitting a pressure adjustment signal with a pressure adjustment signal transmission unit for adjusting an internal pressure of the infusion solution container, and transmitting the pressure adjustment signal based on the internal pressure sensed by the container pressure sensing unit or the oxygen concentration sensed by the oxygen concentration sensing unit.
